(19) United States
(12) Patent Application Publication
(10) Pub. No.: US 2026/0209823 A1
(43) Pub. Date: Jul. 23, 2026

(54) KINASE/PHOSPHATASE SUBSTRATE ANALYSIS AND COMPOSITIONS USING SPECTRALLY ENCODED MICROBEADS

(71) Applicants: CZ Biohub SF, LLC, San Francisco, CA (US); The Board of Trustees of the Leland Stanford Junior University, Stanford, CA (US); University of Copenhagen, Copenhagen (DK)

(72) Inventors: Polly M. Fordyce, Menlo Park, CA (US); Jamin B. Hein, Palo Alto, CA (US)

(21) Appl. No.: 19/138,426

(22) PCT Filed: Dec. 15, 2023
(86) PCT No.: PCT/US2023/084274

§ 371 Date: Jun. 12, 2025

Related U.S. Application Data
(60) Provisional application No. 63/387,757, filed on Dec. 16, 2022.

(51) Int. Cl. C12Q 1/42; C12Q 1/48; G01N 15/14; G01N 33/58

(52) U.S. Cl. CPC C12Q 1/42; C12Q 1/485; G01N 15/14; G01N 33/582; G01N 33/587; G01N 2333/912; G01N 2333/916; G01N 2440/14

(57) ABSTRACT

The disclosure provides methods and compositions for evaluating phosphatase and kinase activity using spectrally encoded microbeads comprising substrate peptides in which there is a 1:1 linkage between sequences of the substrate peptide and the embedded spectral code.

### CROSS REFERENCE TO RELATED APPLICATIONS

This application claims priority benefit of U.S. Provisional Application No. 63/387,757, filed Dec. 16, 2022, which is incorporated by reference for all purposes.

### STATEMENT AS TO RIGHTS TO INVENTIONS MADE UNDER

### FEDERALLY SPONSORED RESEARCH AND DEVELOPMENT

This invention was made with Government support under contract GM123641 awarded by the National Institutes of Health. The Government has certain rights in the invention.

### BACKGROUND

Phosphorylation is one of the most prevalent posttranslational modifications with phosphates added to protein substrates by kinases and removed by phosphatases. A detailed and quantitative understanding of the specificity of these enzymes can help decipher regulatory networks within cells and potentially reveal candidate therapeutic agents with maximal specificity for a particular enzyme target. However, little is known about the sequence determinants of substrate specificity for most kinases and even less is known about phosphatase specificities.

Libraries of spectrally encoded hydrogel beads with >1,000 unique codes (Microspheres with Ratiometric Barcode Lanthanide Encoding (MRBLEs; see, e.g., Gerver et al., Lab on a Chip 2012, Nguyen et al., Adv. Opt. Mat. 2017; and U.S. Pat. No. 10,240,145) and MRBLE production and functionalization processes (see, e.g., Feng et al., eng et al., Microsyst Nanoeng 6: 109, 2020; and WO2002/094219) have been described. It has further been shown that peptides can be synthesized directly on the microbeads with a 1:1 linkage between the peptide sequence and the embedded spectral code. Such bead-bound peptide libraries can be pooled and incubated with fluorescently-labeled proteins to quantify protein/peptide interaction affinities (see, e.g., Nguyen et al., eLife 2019;8:e40499).

### SUMMARY

It is understood that this summary features various aspects of the disclosure and is not provided as a comprehensive summary of all embodiments encompassed by the disclosure.

In one aspect, the disclosure provides methods and compositions for measurement of peptide phosphorylation and dephosphorylation on peptide substrates in parallel (MRBLE_Pak) (FIG. 1A). The small amount of enzyme used for this assay provides the ability to identify substrate specificity parameters with small-scale purifications. Unlike most assays that determine kinetic parameters for a single substrate one at a time or can probe only phosphorylation, but not dephosphorylation, the present methods and compositions provide the ability to efficiently obtain quantitative information about rates of phosphorylation and dephosphorylation.

In some embodiments, the disclosure features methods and compositions that rely on parallel peptide analysis to evaluate dephosphorylation. For example, in phosphatase assays, peptides containing phosphorylated amino acids can be directly synthesized on encoded beads, thereby ensuring that all peptides are site-specifically phosphorylated at desired locations (illustrated in FIG. 1B). Further, inclusion of additional quality control steps, e.g., via mass spectrometry, can verify correct synthesis of phosphorylated peptides.

Thus, in some embodiments of the present disclosure, a pooled bead library bearing phosphorylated peptides with a known linkage between the identity of the peptide and the embedded spectral code is incubated with the phosphatase of interest followed by incubation with one or more reagents to detect peptide-attached phosphates. The spectral signature of the spectrally encoded microbeads can then be determined, e.g., by evaluating the signals emitted from lanthanides embedded in individual microbeads to identify embedded spectral codes, thus identifying the peptide displayed by each bead. In some embodiments, remaining phosphorylated residues are detected using a binding agent that binds to phosphate groups, e.g., titanium dioxide (Ti4+O₂) nanoparticles that bind to phosphates and biotin (see, e.g., the commercially available pIMAGO kit from Tymaro), and fluorescently-labeled streptavidin, which is used to detect peptide-attached biotin. Similarly, unphosphorylated bead-bound peptide libraries can be incubated with kinases of interest and the phosphorylation status can be detected using a similar phosphorylation detection method, e.g., using a phosphate binding agent (e.g., FIG. 1C).

Accordingly, in some aspects of the present disclosure, the disclosure features a method of assaying peptide dephosphorylation comprising providing a population comprising microbeads having distinguishable spectral codes, wherein each microbead displays attached candidate substrate peptides, wherein each substrate peptide contains a phosphorylation site with a phosphorylated amino acid residue, and wherein each microbead having the same spectral code comprises a substrate peptide having a phosphorylation site of the same sequence and microbeads having different spectral codes comprise substrate peptides comprising phosphorylation sites of different sequences; contacting the population of microbeads with a phosphatase; detecting phosphorylated residues of substrate peptides on individual beads following removal of the phosphatase; and determining the spectral signature of individual microbeads in the population, thereby determining the identity of the substrate peptide linked to each bead. In some embodiments, the distinguishable spectral codes are lanthanide spectral codes. In some embodiments, the step of detecting phosphorylated residues comprising incubating microbeads with a labeling reagent that binds to phosphates present on phosphorylated substrate peptides. In some embodiments, the contacting step comprises incubating a pooled population of microbeads with the phosphatase. In some embodiments, the contacting step comprises incubating the microbeads with the phosphatase in the presence of a phosphatase inhibitor or a candidate agent, such as a small molecule, e.g., an organic molecule having a molecular weight from 100-1,000 Da, that modulates phosphatase activity. In some embodiments, the detecting step comprises distributing the pooled population into an array configuration. In some embodiments, the lanthanide spectral codes are detected by deep UV imaging. In some embodiments, the labeled reagent that binds to phosphates comprises a fluorescent label. In some embodiments, the labeled reagent is a biotin-linked titanium dioxide nanoparticle. In some embodiments, phosphates are detected by image analysis. In some embodiments, the phosphorylated substrate peptides are synthesized on the microbeads.

In a further aspect, the disclosure provides a method of assaying substrate peptide phosphorylation comprising providing a population comprising microbeads having distinguishable spectral codes, wherein each microbead has a substrate peptide comprising a phosphorylation site attached thereto and wherein each microbead having the same spectral code displays a substrate peptides comprising the same phosphorylation site sequence and microbeads having different spectral codes comprise substrate peptides comprising phosphorylation sites that differ in sequence; contacting the population of microbeads with a kinase; detecting phosphorylated residues of substrate peptides on individual microbeads; and determining the spectral signature of individual microbeads in the population, thereby determining the identity of the substrate peptide linked to individual beads. In some embodiments, the distinguishable spectral codes are lanthanide spectral codes. In some embodiments, the step of detecting phosphorylated residues comprises contacting microbeads with a labeled reagent that binds to phosphates present on phosphorylated substrate peptides. In some embodiments, the contacting step comprises contacting a pooled population of microbeads with the kinase. In some embodiments, the contacting step comprises contacting the microbeads with the kinase in the presence of a kinase inhibitor or a candidate agent, such as a small molecule, e.g., an organic molecules having a molecular weight from 100-1,000 Da, that modulates kinase activity. In some embodiments, the detecting step comprises distributing the pooled population into an array configuration. In some embodiments, the lanthanide spectral codes are detecting by deep UV imaging. In some embodiments, the labeled reagent that binds to phosphates comprises a fluorescent label. In some embodiments, the labeled reagent is a biotin-linked titanium dioxide nanoparticle. In some embodiments, phosphorylated residues are detected by image analysis. In some embodiments, the substrate peptides are synthesized on the microbeads.

In an additional aspect, the disclosure provides a population of lanthanide-encoded microbeads comprising microbeads having distinguishable lanthanide spectral codes, wherein each microbead comprises a substrate peptide comprising a phosphorylation site attached thereto and wherein each microbead having the same spectral code comprises a substrate peptide comprising the same phosphorylation site sequence and microbeads having different spectral codes comprise substrate peptides comprising phosphorylation sites that differ in sequence.

In another aspect, the disclosure provides a population of lanthanide-encoded microbeads comprising microbeads having distinguishable lanthanide spectral codes, wherein each microbead comprises a phosphorylated substrate peptide comprising a phosphorylation site that contains a phosphorylated amino acid residue, and wherein each microbead having the same spectral code comprises a substrate peptide comprising the same phosphorylation site sequence and microbeads having different spectral codes comprise substrate peptides comprising phosphorylation sites that differ in sequence. In some embodiments, the phosphorylated substrate peptides are synthesized on the microbeads.

### BRIEF DESCRIPTION OF THE DRAWINGS

FIG. 1A-D: Overview of bead-based kinase and phosphatase assays. A) Illustration of phosphorylation dependent signaling events that control protein function. B) Preparation of encoded beads and peptide synthesis; peptides are synthesized on beads such that the embedded spectral code within each bead encodes the identity of the displayed peptide sequence. C ) Assay overview: Pooled encoded beads bearing either unphosphorylated or site-specifically phosphorylated peptides are treated with a kinase or phosphatase of interest, respectively. After incubation, the phosphorylation status of the peptides on the beads is detected using pIMAGO, which attaches biotin molecules to inorganic phosphates. Deep UV imaging is used to decode the beads and thereby identify the underlying peptide sequence; after incubation with fluorescently-labeled streptavidin. Epi-fluorescence imaging quantitatively detects the amount of pIMAGO reagent per bead and thereby the amount of phosphorylated amino acids bound to the beads. D) Experimental setup of phosphatase or kinase assay. Phosphorylated peptides are treated with a phosphatase of interest; or unphosphosphorylated peptides are treated with a kinase of interest.

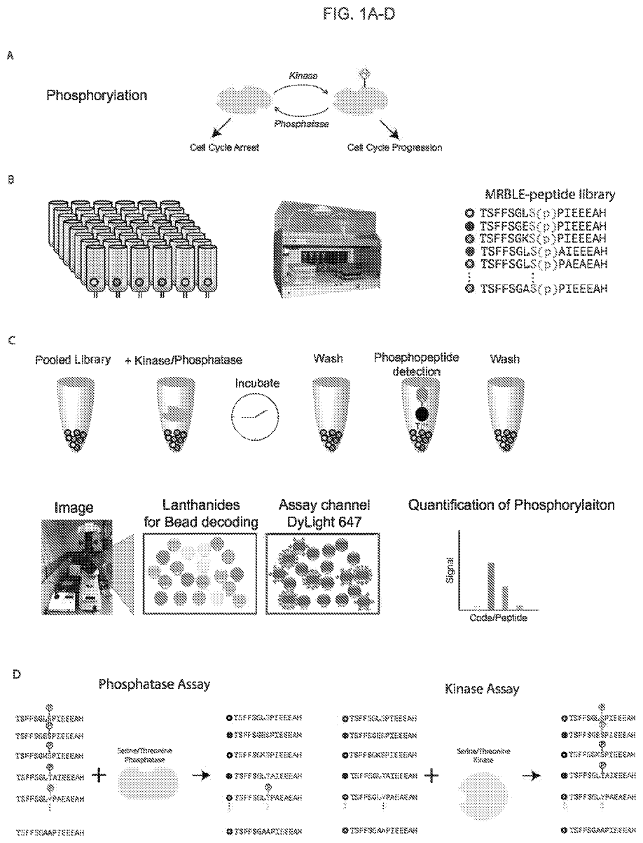

FIG. 2A-B: Overview and example data from bead-based multiplexed phosphatase activity assay. A) Experimental setup of phosphatase assay. Beads with indicated peptides are incubated with either PP1 (top) or Buffer only (bottom). The beads are washed and the phopsphorylation status of the peptides on the beads is detected using pImago. The beads are analyzed using a microscope capable of exciting beads with UV light and then identifying the spectral code embedded within each bead. B) Preliminary data showing the effect of PP1 phosphatase treatment on the phosphorylation status of the TSFFXGISPIEEEAH peptide (X indicates phosphorylated Serine). In the absence of PP1 the pIMAGO reagent binds to phosphorylated Serine, treatment with PP1 removes the phosphate group from Serine, which results in the loss of the signal.

FIG. 3A-C: Overview of using magnetically encoded beads for quality control and validation of peptides synthesized via solid-phase synthesis on beads: A) Desired end-products of the assay: (1) spectrally encoded beads bearing covalently-linked peptides synthesized on beads, and (2) soluble peptides that were synthesized on beads in parallel and can be profiled via mass spectrometry or other techniques for quality control. B) Coupling of Fmoc-Glycine (an acid-resistant linker) to spectrally encoded beads and rink amide F-moc Glycine (an acid-labile linker) to magnetic beads prior to on-bead solid phase peptide synthesis. C) Parallel peptide synthesis on encoded and magnetic beads followed by side chain deprotection using TFA. During exposure to TFA, peptides are deprotected on both bead types; exposure to acid also cleaves the linker linking peptides to magnetic beads, causing them to be eluted from the magnetic bead matrix. Peptides eluted from magnetic beads can then be used for quality control; encoded beads bearing still-attached peptides can be mixed, separated from magnetic beads, and pooled for use in downstream assays.

FIG. 4A-C: Example quality control experiments for on-bead peptide synthesis. A) Experimental overview: After peptide synthesis, peptides are deprotected using standard procedures (e.g., as depicted in FIG. 3). Peptides synthesized on magnetic beads are eluted and can be used for quality control (e.g. by LC/MS). Encoded beads can be easily separated from magnetic beads using a magnet and used for downstream biological assays. In this experiment, the interaction between a protein and a peptide were tested and the interaction analyzed using a specialized microscope. B) Images showing the separation of lanthanide (1) encoded beads and magnetic (m) beads using a magnet. C) LC/MS quality control traces confirming expected identity of eluted peptide.

### DETAILED DESCRIPTION

### Terminology

The term “lanthanide” refers to elements 57-71 of the periodic table, namely lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu). Reference to “lanthanide” can also include combinations of lanthanide elements, compounds containing lanthanide elements or their combinations, or ions containing lanthanide elements or their combinations.

The term “lanthanide nanoparticle” refers to a nanoparticle that includes a lanthanide and a host lattice. Lanthanide nanoparticles are sometimes referred to as “lanthanide nanophosphors”.

The term “host lattice” refers to a material that can accommodate the incorporation of lanthanide atoms or ions. When the host lattice is “lanthanide-doped,” it means that the host lattice material contains one or more lanthanides. For example, lanthanide dopants may be incorporated into a host lattice to provide lanthanide-doped yttrium orthovanadate (YVO4), lanthanide-doped oxides (for example, doped ZrO₂, doped TiO₂, doped BaTiO3), lanthanide-doped halides (for example, doped LaF3), lanthanide-doped phosphates (for example, dope LaPO4, doped LuPO4, or doped YbPO4), and lanthanide-doped strontium borates (for example, SrB4O7, SrB6O10, and Sr4B14O25), among others.

“Lanthanide encoded” or “spectrally encoded” microbeads described in the present disclosure contain lanthanide nanoparticles (Lns) and possess a detectable spectral signature, which is a combination of luminescent signals in the range of 350-850 nm emitted from lanthanide nanoparticles contained in a single microbead upon excitation with an appropriate wavelength of light, for example, UV light (such as 292 nm for excitation of downconverting lanthanides) or IR light (such as 980 nm for excitation of upconverting lanthanides). The luminescence intensity at a characteristic wavelength or wavelengths (for example, 620 nm, 630 nm, or 650 nm) for a particular lanthanide (for example, Eu) indicates the presence and quantity of the particular lanthanide in the source (for example, a microbead) from which the spectral signature originates. A “lanthanide encoded” or “spectrally encoded” microbead may include one or more different types of lanthanide nanoparticles, for example, 2 or more, 3 or more, 4 or more, 5 or more, 6 or more, 7 or more, 8 or more, 9 or more, or 10 or more, wherein each lanthanide nanoparticle has a different luminescence emission spectrum upon excitation. Signals from the combined luminescence spectra make up the spectral signature of a particular microbead, and are mapped to a unique spectral signature code (or “spectral code”) during code deconvolution. Lanthanide nanoparticle spectra are typically characterized by narrow emission bands (also referred to as “signals”) in the visible region, making one species of material easily distinguishable from another. A lanthanide spectral signature of a microbead can therefore be designed based on the particular identity and relative amounts of lanthanides in the microbead. Lanthanide spectral signatures of microbeads described in the present disclosure can include one or more of an Eu signal, a Dy signal, an Sm signal, a Ce signal, a Tb signal, a La signal, a Pr signal, an Nd signal, a Gd signal, an Ho signal, an Er signal, a Tm signal, a Yb signal, a Pm signal, and a Lu signal.

As used herein, the term “microbead” or “microsphere” refers to a particle having one or more dimensions (e.g., length, width, diameter, or circumference) of about 1000 μm or less, e.g., less than about 500 μm, 100 μm, or 10 μm. Microbeads may have a generally spherical shape or a non-spherical shape. Microbeads used in the methods of the present disclosure are characterized by a detectable spectral signature as described in more detail below. A “plurality” of microbeads refers to a population of microbeads ranging in size from a few microbeads to thousands of microbeads, or more. The terms “microsphere” and “microbead” are used interchangeably in the present disclosure regardless of whether the bead has a generally spherical or non-spherical shape.

The term “amino acid” encompasses naturally occurring amino acids as well as non-naturally occurring amino acids, including amino acid analogs and derivatives. Amino acids include naturally occurring proteogenic L-amino acids; D-amino acids; chemically modified amino acids such as amino acid analogs and derivatives; naturally occurring nonproteinogenic amino acids such as norleucine, p-alanine, or ornithine; and chemically synthesized compounds having amino acid characteristics. Amino acids may be referred to herein by either their commonly known three letter symbols or by the one-letter symbols recommended by the IUPAC-IUB Biochemical Nomenclature Commission.

The term “peptide” is used herein to refer to a polymer of amino acid residues. The term applies to amino acid polymers in which one or more amino acid residue is an artificial chemical mimetic of a corresponding naturally occurring amino acid, as well as to naturally occurring amino acid polymers and non-naturally occurring amino acid polymers. Thus, a peptide for used in a panel of peptides to analyze binding of a protein of interest to binding partner peptides as described herein can comprise naturally occurring and/or synthetic amino acids, including analogs and amino acid mimetics that function in a manner similar to the naturally occurring amino acids.

The term “label,” as used herein, refers to any atom or molecule that can be used to provide a detectable and/or quantifiable signal. In some embodiments, the label can be attached, directly or indirectly, to a biomolecule. Labels include, but are not limited to, radioisotopes, fluorophores, chromophores, mass labels, electron dense particles, magnetic particles, spin labels, molecules that emit chemiluminescence, electrochemically active molecules, enzymes, cofactors, and enzyme substrates.

An “array configuration” as used herein refers to a collection of single compartments. An array configuration may be an “ordered array” in which the compartments are addressable and can be assigned to known locations.

A “compartment” as used herein in the context of distributing microbeads refers to any partially or fully enclosed space that separates pools of microbeads or separates individual microbeads. Thus, a compartment can include microwells, microfluidic chambers, and the like.

### Introduction

In some aspects of the present disclosure, a library of peptides comprising phosphorylation/dephosphorylation sites are immobilized to spectrally encoded microspheres with a 1:1 linkage between the peptide sequence and the spectral code to assess kinase and phosphatase activity, e.g., to evaluate specificity of uncharacterized kinases or phosphatases and/or to investigate substrate sequence determinants that influence kinase or phosphate specificity and activity. Substrate peptides typically comprises a serine, threonine or tyrosine residue that is phosphorylated/desphosphorylated, although in some embodiments, a substrate peptide may comprise a histidine, arginine, or lysine residue for phosphorylation/dephosphorylation analysis. Phosphorylation also depends on neighboring amino acids, as detailed below.

Signal generated by a label from a reagent that detects phosphorylation or dephosphorylation events can be assessed for each microbead and the identity of the peptide determined based on the spectral code associated with the signal. In typical embodiments, library populations or subpopulations can be pooled to efficiently assess kinase or phosphatase activities using small amounts of the enzymes.

### Substrate Peptides

In the context of this disclosure, a “substrate peptide” refers to a peptide that comprises a phosphorylation site. A “phosphorylation site” refers to a region of a peptide that comprises a residue, which, in the context of a short sequence recognition motif, can be phosphorylated by a kinase and/or dephosphorylated via activity of a phosphatase. As indicated above, substrate peptides typically comprise a serine, threonine or tyrosine residue that is phosphorylated/dephosphorylated. In some embodiments, a substrate peptide can comprise a histidine or arginine that is phosphorylated. In other embodiments, a substrate peptide comprises a lysine residue for phosphorylation/desphosphorylation analysis.

In some embodiments, a substrate peptide sequence is from a naturally occurring peptide that is phosphorylated/dephosphorylated. Such substrate regions can be identified, e.g., using a variety of databases, e.g., PhosphoAtlas, Phospho. EM, PhosphoNET, PHOSIDA, PhosphoPep, and PhosphoSitePlus databases, among others (see, e.g., Olow et al., “An Atlas of the Human Kinome Reveals the Mutational Landscape Underlying Dysregulated Phosphorylation Cascade in Cancer” Cancer Res. 76(7):1733-45, Apr. 1, 2016; Epub 2016 February 26; and Hornbeck et al., “PhosphositePLus, 2014: Mutations, PTMs and recalibrations. Nucl. Acids Res. 43:D 512-D520, 2015; Phospho. ELM, Dinkel et al., Nucl. Acid Res. 39, Issue suppl_1, 1 Jan. 2011: D261-D267,)

In some embodiments, the substrate peptide may be a “synthetic” peptide, which as used in this context, refers to a peptide designed to test kinase or phosphatase activity and/or specificity, but that does not contain a native phosphorylation/dephosphorylation sequence that has been identified in a protein that undergoes kinase-dependent phosphorylation and/or phosphatase-dependent dephosphorylation.

In some embodiments, a diversity of synthetic peptides can be generated based on consensus sequences or motif sequences of naturally occurring sites that are phosphorylated/dephosphorylated (see, e.g., Miller & Turk, Methods Mol Biol 1360:203-216, 2106, Bradley & Beltrao et al, PLoS Biol 17(6): e3000341, and references cited therein, as well as phosphorylation site databases, e.g., cited above). In some embodiments, synthetic peptide sequences can be generated based on bioinformatics tools, including deep learning methods to predict kinase-specific phosphorylation sites. Such resources are available, see, e.g., Wang et al, Genomics, Proteomics & Bioinformatics 18:72-80, 2020; Trost et al., Bioinformatics 27:2927-2935, 2011; Song et al, Sci Rep. 7:2862, 2017; Kirchoff et al, Bioinformatics 38:2119-2126, 2022; Ma et al, Genomics, Proteomics & Biotinformatics, 2022, doi: 10.1016/j.gpb.2022.06.004, Novacek et al, PLOS Comput. Biol 16:e1007578, 2020; PhosphoMotif Finder, ScanProsite, and many others.

In some embodiments, a synthetic peptide may be generated based on introducing selected or random mutations into a known kinase-dependent phosphorylation site to alter amino acid residues in sequences that flank the residue that is phosphorylated, e.g., alter residues that are adjacent to, or 2, 3, 4, or 5 amino acids N-terminal or C-terminal of the phosphorylated residue. In some embodiments, mutations are introduced that have been identified as influencing phosphorylation in a disease state such as cancer. See for example, mutations associated with altered phosphorylation sites reviewed by Reimand & Bader in Mol Syst Biol. 9: 637, 2013; Huang et al, Nat Commun 12, 2313, 2021.

Substrate peptides can vary in length, e.g., from 7 to 25 amino acids in length, or from 9 to 21 amino acids in length. In some embodiments, a peptide may be up to 50 amino acids in length, or even up to 100 amino acids in length and at least 5 amino acids in length. In some embodiments, a substrate peptide is 9, 10, 11, or 12 amino acids in length. In some embodiments, substrate peptides can comprise additional amino acids at the C-terminal and/or N-terminal ends of the peptide.

Various control substrate peptides may also be represented in the population of microspheres comprising linked substrate peptides, e.g., for experimental controls and statistical analysis, including generic positive controls and/or peptides or random sequences, e.g., with a similar or identical amino acid content.

In some embodiments, a population of microspheres as described herein may include substrate peptides to any number of kinases and/or kinase family members, including, for example, to evaluate specificity of individual kinases that are members of kinase families or subfamilies. In some embodiments, such kinases can include family members that are dysregulated in disease states, such as cancer. In some embodiments, substrate specificity can be evaluated for kinases of various kinase families, including ABL, SRC, JAK, PTK2/FAK, EGFR, FGFR, FLT/VEGFR, IGFR/INSR, PDGFR, ROR, EPHA/B, ALK, AKT, RAF, AURK, CHEK, PLK, BRSK, CAMK, CDK, IKK, CLK, CK, IRAK, EIF2AK, GRK, GSK3, ERK, JNK, p38 MAP, MEK, MAP3K, MAPKAPK, MARK, NEK, NME, PAK, PIM, PKN, AMPKa, PKA, PKC, PKD, PKG, ROCK, RPS6KA, RPS6KB, SGK, or TSSK families.

In some embodiments, a population of substrate peptides linked to spectrally encoded microsphere as described here are used to evaluate phosphatase activity. Phosphatases are often considered to fall into two categories: serine/threonine phosphatases and tyrosine phosphatases. In some cases, dual specificity tyrosine phosphatase can dephosphorylate other residues, such as serine. In some embodiments in which phosphatase activity is evaluated, substrate peptides linked to microspheres are synthesized on the bead with phosphorylated residues at the phosphorylation site.

A kinase or phosphatase to be evaluated may be a purified or partially purified preparation or may be present in a protein sample obtained from an organism. Thus a sample comprising a kinase or phosphatase activity can be obtained from cells or tissues of any prokaryote or eukaryote, including plants, yeast, bacteria, cyanobacteria, or any other biological source of interest.

In some embodiments in which phosphatase activity is evaluated, substrate peptides linked to microspheres are synthesized on the bead with phosphorylated residues at the phosphorylation site.

### Synthesis of Peptides on Microbeads

Synthesis of peptide directly on microbeads is known. See, for example, the methods detailed in WO2021252735 and WO2022094219, each incorporated by reference.

In brief, such methods allow for inclusion of reactive (or functional) groups on a surface of the polymeric microspheres during microsphere solidification, i.e., the lanthanide-encoded polymeric microbeads can be functionalized as a part of the production process without adding additional steps after microbeads are performed. Functionalization of lanthanide-encoded polymeric microbeads can be accomplished by including a suitable amphipathic compound in a second, immiscible fluid used for droplet generation. Such a suitable amphipathic compound is capable of covalently bonding with the microbead matrix component during the solidification step. A suitable amphipathic compound includes one or more reactive groups that remain free after covalent binding of the amphipathic compound to the surfaces of the polymeric microbeads, and these free reactive groups can be used for subsequent attachment of molecules or moieties of interest to the microbeads, e.g., a binding moiety. Molecules of the suitable amphipathic compound included in the continuous phase are driven to and remain at the interface of the immiscible fluid and the matrix component-and lanthanide particle-containing fluid after droplet formation, with the hydrophobic parts of the amphipathic molecules facing a hydrophobic fluid (which may be the matrix and nanoparticle-containing fluid or the immiscible fluid), and the hydrophilic parts of the amphipathic molecules facing a hydrophilic fluid (which may be the matrix and nanoparticle-containing fluid or the immiscible fluid). In some embodiments, polymeric microbeads can be functionalized by two or more different reactive groups during their production by using, for example, an amphipathic compound with two or more reactive groups. In another example, multiple amphipathic compounds with different reactive groups can be added to the continuous phase during droplet generation.

In other embodiments, microbeads having various functional groups that can be used for on-bead synthesis or chemical coupling of peptide are generated using a technique described by Feng et al., Microsyst Nanoeng 6:109, 2020, which is incorporated by reference. This method employs mixing of lanthanide-polymer mixture followed by droplet generation using a single-layer, parallel flow-focusing device, with polymerization of droplet in batch off of the chip. Copolymers bearing functional groups typically used for bioconjugation are localized to the surface of the hydrogel matrix during droplet generation and these polymers are covalently cross-linked in place during bead polymerization.

Some non-limiting examples of compounds that can be used for microbead functionalization and included in the second fluid are: for functionalization with carboxyl groups, unsaturated fatty acids, such as 10-undecenoic acid, 4-pentenoic acid, 5-hexenoic acid, 6-heptenoic acid, 7-octenoic acid, 8-nonenoic acid, or 9-decenoic acid; for functionalization with amino groups, amphiphathic amines, such as pent-4-enylamine, N-(3-Aminopropyl)methacrylamide, 2-Aminoethyl methacrylate, or N-(2-aminoethyl) methacrylamide; for functionalization with azide groups, 3-azidopropyl acrylate or 3-azidopropyl methacrylate; for functionalization with hydroxyl groups, 4-Penten-1-ol; for functionalization with hydrazide groups, hydrazido acrylate; for functionalization with chloromethyl groups, chloromethyl acrylate.

In some embodiments, a reactive group comprises an amino group, and the method further comprises performing solid-phase peptide synthesis on a surface of the polymeric microbeads. Thus, for example, in some embodiments, beads having a specific spectral code signature are collected into a single compartment, e.g., a well of a microwell plate, and the plate is transferred to a high-throughput solid phase peptide synthesizer to synthesize peptides on beads with a 1:1 linkage between the bead spectral code and substrate peptide. The direct incorporation of the reactive group, e.g., pent-4-eylamine) during bead production ensures that the bead-peptide linkage is covalently coupled to the rest of the matrix and remains stable during peptide synthesis and side chain deprotection.

In some embodiments, magnetic polymeric beads with the same chemical composition as the spectrally encoded beads, but having an acid-labile linker, are incorporated into the bead pool used for solid phase synthesis. The beads can then be separated from the rest of the pool using a magnet and the material eluted for downstream characterization, e.g., by mass spectrometry and quality control.

In some embodiments, substrate peptides comprising phosphorylated residues are synthesized directly on microbeads for evaluation of phosphatase activity.

In some embodiments, a reactive group comprises a hydroxyl group, and the method further comprises performing cyanogen bromide-activated covalent coupling of an amino acid, a peptide or a protein to the reactive group. In some exemplary methods for producing derivatized microbeads, a reactive group comprises a hydroxyl group, and the method further comprises performing cyanogen bromide-activated covalent coupling of an amino acid, a peptide or a protein to the reactive group. In some exemplary methods for producing derivatized microbeads, a reactive group comprises a chloromethyl group, and the method further comprises performing covalent coupling of an amino acid, a peptide, a protein or other amino-group containing molecule to the reactive group. In some exemplary methods for producing derivatized microbeads, a reactive group comprises an azide group, and the method further comprises performing covalent coupling of a molecule or moiety of interest via click chemistry to the reactive group.

In other embodiments, biotin-modified peptides may be loaded onto the microbeads via interaction with streptavidin coated on the surface of the spectrally encoded microbeads.

In some embodiments, beads are distributed to one or more compartment for kinase or phosphatase activity assessment. In some embodiments, pools of substrate peptide-linked microbeads are pooled for kinase or phosphatase activity assay.

### Spectral Signature

In general, each of the spectral signatures employed in an assay contains signals generated from predetermined amounts of two or more lanthanides (e.g., two or more Eu-, Dy-, Sm-, Ce-, Tb-, La-, Pr-, Nd-, Gd-, Ho-, Er-, Tm-, Yb-containing materials such as nanoparticles). Lanthanide materials in the microbeads can be excited with UV light (e.g., 275 nm or 292 nm) and emitted luminescent signals can be detected in the range of 400-800 nm (e.g., 435 nm, 474 nm, 527 nm, 536 nm, 546 nm, 572 nm, 620 nm, 630 nm, 650 nm, or 780 nm). A signal in a spectral signature may be measured as an absolute value or as a ratio of the signal to another reference signal. As a non-limiting example, a set of unique spectral signatures can be prepared with microparticles that contain europium-doped yttrium orthovanadate (YVO₄:Eu) to generate a reference signal and varying amounts of YVO₄:Dy, YVO₄:Sm, YVO₄:Tm, and LaPO₄:CeTb.

In some embodiments, deep UV imaging can be used to determine spectral signatures of microbeads. For example, in some embodiments, a substrate-peptide-linked beads are distributed in an array configuration before or after kinase or phosphatase incubation, a spectral signature of a microbead can be determined by imaging, e.g., deep UV imaging for lanthanides.

In some embodiments, lanthanides embedded in different host matrices excite at low energy light, e.g., 980 nm IR, and emit in visible light. In applications employing such “up-converting” lanthanide: host matrix combinations, the spectral signature is determined via infrared imaging.

In some embodiment, lanthanide signatures are determined for each bead that undergoes a phosphorylation or dephosphorylation event in the assay.

### Kinase/phosphatase Activity

Kinase activity can be evaluated using any number of assays, including, e.g., measuring ATP consumption; measuring consumption of any factor related to catalytic activity of an enzyme (e.g. GTP for GTPases instead of ATP for ATP-dependent kinases) or by physical detection of the catalytic addition or removal of a phosphate group onto a substrate peptide, e.g., using an antibody or other phosphate-binding agent, such as titanium dioxide nanoparticles, that bind to phosphate. Activity can be evaluated through detection of a signal generated from a detectable label from one or more reagents used to detect phosphates.

In one illustrative assay, phosphorylated substrate peptides can be synthesized directly on microbeads to ensure that all peptides are phosphorylated. Beads are pooled and then incubated with a phosphatase of interest, washed, and incubated with reagents to detect a phosphate retained by the substrate peptide. Beads are evaluated, typically by image analysis, to identify the embedded spectral code (i.e., spectral signature) of each bead and the amount of remaining phosphorylation quantified by measuring the level of signal generated from a phosphate detection reagent. Kinase activity is similarly assessed, but using by using unphosphorylated substrate peptides synthesized directly on microbeads.

A “sample” for assay of kinase or phosphatase activity can be a purified, or partially purified kinase or phosphatase preparation or can be an extract prepared from a biological source, such as tissues or cells.

In some embodiments, kinase activity towards peptide substrates is assessed in the presence of a known kinase inhibitor used therapeutically, e.g., for the treatment of cancer (see, e.g., reviews by Kannaiyan & Mahadevan, Expert Rev Anticancer Ther. 12:1249-1270, 2018; and Cohen et al., Nat Rev Drug Discovery 20:551-569, 2021).

In some embodiments, kinase or phosphatase activity towards peptide substrates is assessed in the presence of a candidate inhibitor, such as a small molecule. A “small molecule” as used herein includes, but is not limited to, organic or inorganic compounds that typically have a molecular weight of less than about 5,000 Da. In some embodiments, the small molecule is a small organic compounds that has a molecular weight of less than 1,000 Da. In some embodiments, the agent is a small molecule having a molecular weight of about 100 to about 1,000 Da or about 500 to about 5,000 Da. As used here, “about” refers to a range within 10% or within 20% of the indicated value.

### Techniques to Further Illustrate Aspects of the Disclosure

In some embodiments, highly monodisperse beads comprised of a PEG-DA hydrogel matrix using microfluidic droplet generators are produced (e.g., Feng et al., Microsystems & Nanoengineering 2020). These beads can be spectrally encoded via the ratiometric incorporation of different lanthanide nanophosphors and functionalized with amino groups. In the present disclosure, lanthanides are advantageously used for spectral signatures; in some embodiments however, e.g., using small libraries with a small number of substrate peptides, e.g., fewer than 200, other agents that generate a color signal, such as fluorescent molecules, may be embedded in microbeads for spectral encoding.

After production, beads containing a given code are collected in a single compartment, e.g., a well in a microwell plate, such as a 96-well plate, so that the entire plate can be transferred to a high-throughput solid-phase peptide synthesizer to synthesize peptides on beads with a 1:1 linkage between the peptide sequence and the embedded spectral code. Direct incorporation of the chemical functional reagent for linking an amino group (e.g., pent-4-enylamine) during bead production ensures that the bead-peptide linkage is covalently coupled to the rest of the hydrogel matrix and remains stable during peptide synthesis and side-chain deprotection. After peptide synthesis, the beads can be pooled for efficient assay of kinase or phosphatase activity. In alternative embodiments, beads can be distributed in an array configuration.

An assay to measure dephosphorylation is performed by incubating the (phospho) substrate peptides, e.g., synthesized directly on the encoded beads, with a phosphatase of interest. After a desired time, the reaction is stopped by adding a phosphatase inhibitor, the beads are washed, and then imaging reagents, e.g., pIMAGO reagents from Tymora are added to provide unbiased detection of phosphorylation status of the peptides on the encoded beads. After a wash to remove pIMAGO detection reagents, beads can be imaged, e.g., using a microscope that facilitates both standard epifluorescence imaging (to identify the degree of phosphorylation of beadbound peptides) and lanthanide imaging (excitation in the deep UV along with light collection in 9 narrow distinct emission bands). These images can then be analyzed using open-source image analysis software, e.g., Harink et al., PLOS ONE 2019, to identify the code (and thus the peptide sequence) and amount of phosphorylation associated with each bead. The ratio of phosphorylation detected between a control reaction in the absence of the phosphatase and in the presence of the phosphatase allows determination of how efficiently different peptide sequences are dephosphorylated. Kinases of interest are evaluated using essentially the same assay, but using unphosphorylated substrate peptides to detect kinase activity. One of skill understands that alternative phosphate detection reagents, e.g., antibodies can be employed to determine the presence of phosphates. Similarly, any detection method that detects spectral signals from the encoded microbeads can be employed.

One of skill additionally understands that the assays described herein for kinase and phosphatase activity analysis can be adapted to evaluate catalytic activity of other enzymes that can act on a peptide substrate.

### Example 1. Protein Phosphatase 1 Assay

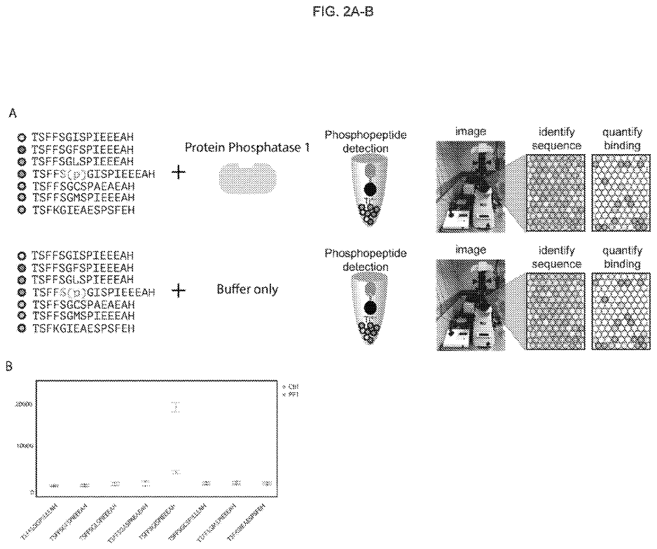

Reversible protein phosphorylation is one of the most important post-translational modifications to regulate signal transduction pathways. Protein phosphatase 1 (PP1) is a major serine/threonine protein phosphatase important for the regulation of various cellular process. We synthesized a panel of eight peptides in which one peptide contains a phosphorylated Serine residue. The phosphorylation status of the encoded beads was analyzed using pIMAGO. The signal of the phosphorylated peptide was markedly reduced after the addition of PP1 (FIG. 2).

### Example 2. Quality Control for On-Bead Peptide Synthesis

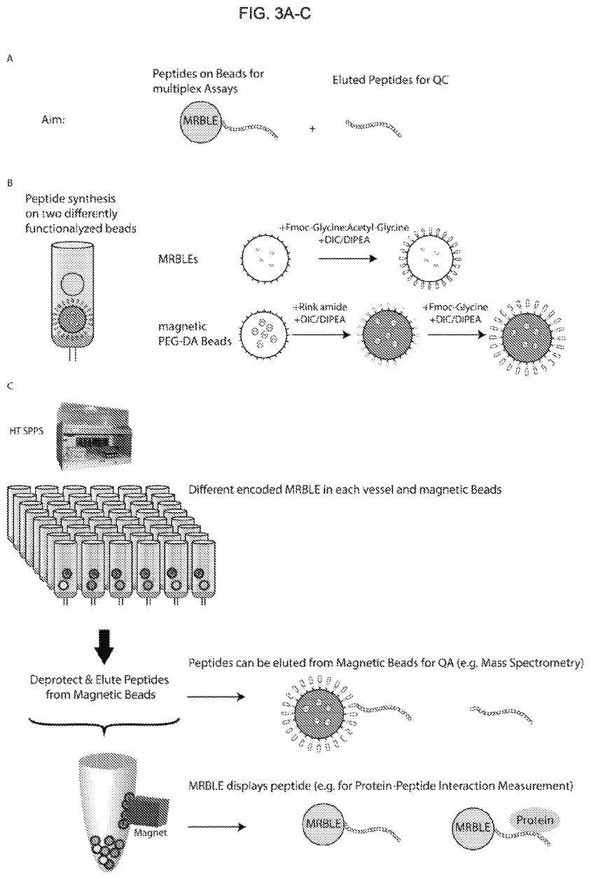

FIG. 3A-C illustrates a method for quality control for on-bead peptide synthesis. As described above, a microfluidic chip is used to produce beads by embedding the lanthanide nanphosphors in a PEG-DA hydrogel matrix with pent-4-enylamine (as a chemical handle for peptide synthesis). Magnetic beads using Fe₃O₄ nanoparticles instead of lanthanide for embedding into a PEG-DA hydrogel functionalized with pent-4-enylamine are also prepared. The magnetic beads are isolated from the lanthanide-encoded beads using a magnet. Prior to peptide synthesis an acid labile linker (Rink amide) is attached to the magnetic beads in order to cleave the peptides of the beads during the global side chain deprotection step after peptide synthesis. In particular, amine-functionalized magnetic beads are treated with DIC/DIPEA to attach rink amide as an acid labile linker. Subsequently, Fmoc-Glycine is attached to amine functionalized encoded beads and the rink amide magnetic beads. The encoded and magnetic beads are mixed, and peptides are synthesized on an automated solid-phase peptide synthesizer (FIG. 3C). After completion the side protecting groups of the peptides are removed using a mixture of TFA, TIPS, and H2O. This treatment also results in the selective cleavage of the peptides attached to the magnetic beads. These peptides are collected, washed and analyzed using LC/MS to verify the identity of eluted peptides.

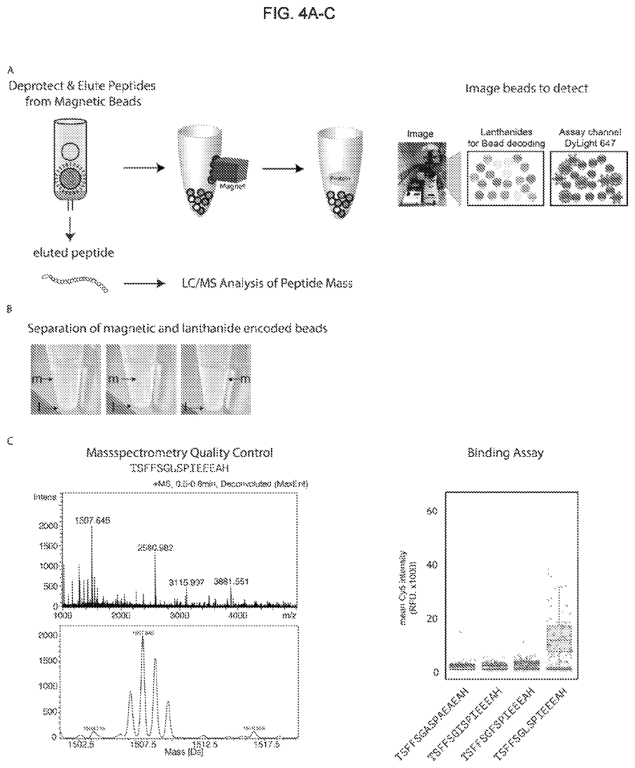

An example of LC/MS quality control for interaction of a peptide and a B56 family protein phosphatase 2A (PP2A) is provided in FIGS. 4A-C. After peptide synthesis, peptides were deprotected using standard procedures. Peptides synthesized on magnetic beads were eluted and used for quality control by LC/MS. Encoded beads were separated from magnetic beads using a magnet and used for downstream biological assays. In this experiment, the interaction between the B56 protein and the peptide (shown in FIG. 4C) was tested and the interaction analyzed using a specialized microscope. FIG. 4B provides images showing the separation of lanthanide (1) encoded beads and magnetic (m) beads using a magnet. LC/MS quality control traces confirming expected identity of eluted peptide are shown in FIG. 4C. Fluorescent signal resulting from binding of peptides displayed on spectrally encoded beads is shown in the right panel of FIG. 4C. The assay was performed using a solution with 500 nM Alexa 647-labeled anti-mouse antibody, 500 nM anti B56 antibody and 1 uM recombinant B56 protein pre-incubated for 1 hour and then added to the encoded beads overnight. The fluorescent signal that shows protein binding to the peptides on the beads and the lanthanide luminescence to decode the beads was detected using a deep UV and fluorescence microscope.

It is understood that the examples and embodiments described herein are for illustrative purposes only and that various modifications or changes in light thereof will be suggested to persons skilled in the art and are to be included within the spirit and purview of this application and scope of the appended claims.

1. A method of assaying peptide dephosphorylation comprising providing a population comprising microbeads having distinguishable spectral codes, wherein each microbead comprises a substrate peptide attached thereto, wherein each substrate peptide comprises a phosphorylation site that contains a phosphorylated amino acid residue, and wherein each microbead having the same spectral code comprises a substrate peptide having a phosphorylation site of the same sequence and microbeads having different spectral codes comprise substrate peptides comprising phosphorylation sites of different sequences;
contacting the population of microbeads with a phosphatase;
detecting phosphorylated residues of substrate peptides on individual beads following removal of the phosphatase;
determining the spectral signature of individual microbeads in the population, thereby determining the identity of the substrate peptide linked to each bead.

2. The method of claim 1, wherein the distinguishable spectral codes are lanthanide spectral codes.

3. The method of claim 2, wherein the step of detecting phosphorylated residues comprises contacting microbeads with a labeled reagent that binds to phosphates present on phosphorylated substrate peptides.

4. The method of claim 1, wherein the contacting step comprises detecting a pooled population of microbeads with the phosphatase.

5. The method of claim 4, wherein the detecting step comprises distributing the pooled population into an array configuration.

6. The method of claim 1, wherein the lanthanide spectral codes are detecting by deep UV imaging.

7. The method of claim 1, wherein the labeled reagent that binds to phosphates comprises a fluorescent label.

8. The method of claim 1, wherein the labeled reagent is a biotin-linked titanium dioxide nanoparticle.

9. The method of claim 1, wherein phosphates are detected by image analysis.

10. The method of claim 1, wherein the phosphorylated substrate peptides are synthesized on the microbeads.

11. A method of assaying substrate peptide phosphorylation comprising providing a population comprising microbeads having distinguishable spectral codes, wherein each microbead comprises a substrate peptide comprising a phosphorylation site attached thereto and wherein each microbead having the same spectral code comprises a substrate peptide comprising the same phosphorylation site sequence and microbeads having different spectral codes comprise substrate peptides comprising phosphorylation sites that differ in sequence;
contacting the population of microbeads with a kinase;
detecting phosphorylated residues of substrate peptides on individual microbeads;
determining the spectral signature of individual microbeads in the population, thereby determining the identity of the substrate peptide linked to individual beads.

12. The method of claim 11, wherein the distinguishable spectral codes are lanthanide spectral codes.

13. The method of claim 11, wherein the step of detecting phosphate residues comprises contacting microbeads with a labeled reagent that binds to phosphates present on phosphorylated substrate peptides.

14. The method of claim 11, wherein the contacting step comprises contacting a pooled population of microbeads with the kinase.

15. The method of claim 14, wherein the detecting step comprises distributing the pooled population into an array configuration.

16. The method of claim 11, wherein the lanthanide spectral codes are detecting by deep UV imaging.

17. The method of claim 11, wherein the labeled reagent that binds to phosphates comprises a fluorescent label.

18. The method of claim 1, wherein the labeled reagent is a biotin-linked titanium dioxide nanoparticle.

19. The method of claim 1, wherein phosphates are detected by image analysis.

20. The method of claim 1 wherein the phosphorylated substrate peptides are synthesized on the microbeads.

21. A population of lanthanide-encoded microbeads comprising microbeads having distinguishable lanthanide spectral codes, wherein each microbead comprises a substrate peptide comprising a phosphorylation site attached thereto and wherein each microbead having the same spectral code comprises a substrate peptide comprising the same phosphorylation site sequence and microbeads having different spectral codes comprise substrate peptides comprising phosphorylation sites that differ in sequence; or
a population of lanthanide-encoded microbeads comprising microbeads having distinguishable lanthanide spectral codes, wherein each microbead comprises a phosphorylated substrate peptide comprising a phosphorylation site that contains a phosphorylated amino acid residue, and wherein each microbead having the same spectral code comprises a substrate peptide comprising the same phosphorylation site sequence and microbeads having different spectral codes comprise substrate peptides comprising phosphorylation sites that differ in sequence.

22. (canceled)

23. The population of claim 21, wherein the phosphorylated substrate peptides are synthesized on the microbeads.
